(19) United States
(12) Patent Application Publication
(10) Pub. No.: US 2025/0348361 A1
(43) Pub. Date: Nov. 13, 2025

(54) Data Management Device and Data Management Method

(71) Applicant: SHIMADZU CORPORATION, Kyoto-shi (JP)

(72) Inventors: Tomonori OSHIKAWA, Kyoto-shi (JP); Xusheng ZHANG, Kyoto-shi (JP); Shiori NAGAI, Kyoto-shi (JP); Kenta ADACHI, Kyoto-shi (JP); Tomoya TSUDA, Kyoto-shi (JP)

(21) Appl. No.: 19/200,306

(22) Filed: May 6, 2025

(30) Foreign Application Priority Data
May 10, 2024 (JP) 2024-077393

(51) Int. Cl. G06F 9/50

(52) U.S. Cl. CPC G06F 9/5027

(57) ABSTRACT

A memory of a data management device stores: two or more types of processing modules; and module information including character strings each corresponding to each of the two or more types of processing modules. A processor of the data management device is configured to select, among the two or more types of processing, for given analysis data, one processing module to be used for processing the given analysis data based on the module information and a character string included in a file name of the given analysis data.

### CROSS REFERENCE TO RELATED APPLICATIONS

This nonprovisional application is based on Japanese Patent Application No. 2024-077393 filed on May 10, 2024 with the Japan Patent Office, the entire contents of which are hereby incorporated by reference.

### BACKGROUND OF THE INVENTION

### Field of the Invention

The present invention relates to management of data of each of a plurality of materials.

### DESCRIPTION OF THE BACKGROUND ART

There has been proposed a system for transversely managing a plurality of pieces of analysis data obtained by a plurality of types of analyzers. As an example of this type of system, Japanese Patent Laying-Open No. 2017-194360 discloses a sample data management system. The sample data management system acquires measurement data of a target sample by using a plurality of types of analyzers including: at least one of an X-ray fluorescence spectrometer, an atomic absorption spectrophotometer, and an inductively coupled plasma atomic emission spectrometer; and at least one of an infrared spectrophotometer and a Raman spectrophotometer, and then specifies the target sample based on the acquired measurement data.

### SUMMARY OF THE INVENTION

In research and development of new materials, a plurality of materials are prototyped by preparing a plurality of raw materials and adjusting a synthesizing and processing process. Further, the performance of each prototype material is evaluated, and if the desired performance is not achieved, the prototype material is analyzed for cause investigation. In the analysis of the prototype material, a part of the material is scraped off or cut out by an appropriate method, and the obtained part of the material is directly measured by various analyzers or is dissolved in a solvent to obtain a sample which is then measured by various analyzers. Hereinafter, a prototype material is simply referred to as a material. Since a plurality of types of analysis data are obtained from one material and the respective pieces of analysis data are acquired by different devices, the respective pieces of analysis data are processed also by different processing modules.

In the system as described above, a device for managing data may require processing of analysis data acquired in each of a plurality of types of analyzers in order for a user to use the device to check the analysis data acquired in each of the plurality of types of analyzers. In such a case, in order to allow the device for managing data to specify a program to be used for processing each piece of analysis data, the information specifying a program to be used for processing each piece of analysis data needs to be registered for each piece of analysis data. However, the operation to register such information is cumbersome and involves a risk of registering incorrect information.

The present invention has been made in view of the above circumstances, and an object thereof is to provide a technique for, in a device that processes analysis data acquired in a plurality of types of analyzers, simply and accurately registering information specifying a program to be used for processing each piece of analysis data.

A data management device according to an aspect of the present disclosure is a data management device that manages analysis data of a material. The data management device includes: a memory; and a processor accessible to the memory. The memory stores: two or more types of processing modules; and module information including character strings each corresponding to each of the two or more types of processing modules. The processor selects, among the two or more types of processing modules, for given analysis data, one processing module to be used for processing the given analysis data, based on the module information and a character string included in a file name of the given analysis data.

A data management method according to an aspect of the present disclosure is a method implemented by a computer for managing analysis data of a material. The computer is configured to process the analysis data by using two or more types of processing modules, and includes a memory that stores module information including character strings each corresponding to each of the two or more types of processing modules. The method includes: reading a file name of given analysis data; and, based on the module information and a character string included in the file name, selecting, among the two or more types of processing modules, one processing module to be used for processing the given analysis data.

The foregoing and other objects, features, aspects, and advantages of the present invention will become apparent from the following detailed description of the present invention when taken in conjunction with the accompanying drawings.

### BRIEF DESCRIPTION OF THE DRAWINGS

FIG. 1 is a schematic diagram illustrating a configuration example of a data management system according to the present embodiment.

FIG. 2 is a diagram schematically showing a hardware configuration example of an information processing device 6 and a data management device 1.

FIG. 3 is a diagram showing an example of a data structure of a storage unit 11 in data management device 1.

FIG. 4 is a diagram showing an example of a data structure of a database 15 in data management device 1.

FIG. 5 is a diagram showing a specific example of configuration information of each of projects.

FIG. 6 is a diagram showing an example of a display screen of a management application.

FIG. 7 is a diagram showing another example of the display screen of the management application.

FIG. 8 is a diagram showing still another example of the display screen of the management application.

FIG. 9 is a diagram showing still another example of the display screen of the management application.

FIG. 10 is a diagram showing still another example of the display screen of the management application.

FIG. 11 is a diagram showing still another example of the display screen of the management application.

FIG. 12 is a diagram showing still another example of the display screen of the management application.

FIG. 13 is a diagram showing still another example of the display screen of the management application.

FIG. 14 is a diagram showing still another example of the display screen of the management application.

FIG. 15 is a diagram showing still another example of the display screen of the management application.

FIG. 16 is a diagram showing still another example of the display screen of the management application.

FIG. 17 is a diagram showing still another example of the display screen of the management application.

FIG. 18 is a flowchart of a process performed by execution of the management application by data management device 1.

FIG. 19 is a flowchart of a subroutine of a process of registering a new project.

FIG. 20 is a flowchart of a subroutine of a process of registering a new material.

FIG. 21 is a flowchart of a subroutine of a process of registering analysis data.

FIG. 22 is a flowchart of a subroutine of a process of displaying the analysis data.

FIG. 23 is a flowchart of a subroutine of a process of searching the analysis data.

FIG. 24 is a flowchart of a modification of the process in FIG. 21.

### DESCRIPTION OF THE PREFERRED EMBODIMENTS

Hereinafter, embodiments of the present invention will be described in detail with reference to the accompanying drawings. In the following description, the same or corresponding portions in the drawings are denoted by the same reference characters, and the description thereof will not be repeated in principle.

### Configuration Example of Data Management System

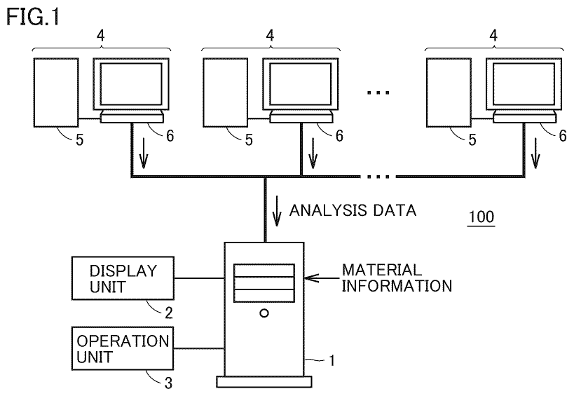

FIG. 1 is a schematic diagram illustrating a configuration example of a data management system according to the present embodiment. As shown in FIG. 1, a data management system 100 according to the present embodiment includes a data management device 1 and a plurality of analyzers 4.

Analyzer 4 generates analysis data by measuring a material and analyzing the result of the measurement. Analyzer 4 may be, for example, a liquid chromatograph (LC), a gas chromatograph (GC), a liquid chromatograph-mass spectrometer (LC-MS), a gas chromatograph-mass spectrometer (GC-MS), a pyrolysis gas chromatograph-mass spectrometer (Py-GC/MS), a scanning electron microscope (SEM), a transmission electron microscope (TEM), an energy dispersive X-ray fluorescence spectrometer (EDX), a wavelength dispersive X-ray fluorescence spectrometer (WDX), a nuclear magnetic resonator (NMR), or a Fourier transform infrared spectrophotometer (FT-IR).

Analyzer 4 may also be a photodiode array detector (LC-PDA), a liquid chromatograph tandem mass spectrometer (LC/MS/MS), a gas chromatograph tandem mass spectrometer (GC/MS/MS), a liquid chromatograph ion-trap time-of-flight mass spectrometer (LC/MS-IT-TOF), a near-infrared spectrometer, a tensile tester, a compression tester, an atomic optical emission spectrometer (AES), an atomic absorption spectrometer (AAS/FLAAS), a plasma mass spectrometer (ICP-MS), an organic element analyzer, a glow discharge mass spectrometer (GDMS), a particle composition analyzer, a trace total nitrogen analyzer (TN), a highly sensitive nitrogen-carbon analyzer (NC), or a thermal analyzer.

Since data management system 100 includes a plurality of different types of analyzers 4, data management device 1 can manage a plurality of types of analysis data related to one material.

Analyzer 4 includes a device main body 5 and an information processing device 6. Device main body 5 measures a sample to be analyzed. The identification information of the sample and the measurement condition for the sample are input to information processing device 6.

Information processing device 6 controls the measurement in device main body 5 according to the input measurement condition. Thereby, analysis data based on the result of measurement of the sample is acquired. Information processing device 6 incorporates a memory. Information processing device 6 causes the memory to store the measurement result of the sample as analysis data together with the identification information, the measurement condition, and the analysis result for the sample. For example, when analyzer 4 is LC-MS, the measurement result includes a count value at each m/z, the identification information of the sample includes identification information imparted to the sample as a measurement target in data management system 100, the measurement condition includes a setting parameter (for example, the gas flow rate of an ion source unit) for the LC-MS used when the measurement result is obtained, and the analysis result includes items (for example, a peak area) derived by analyzing the measurement result.

Information processing device 6 is communicably connected to data management device 1. Thereby, information processing device 6 of each analyzer 4 can transmit the analysis data of each sample to data management device 1. Information processing device 6 and data management device 1 may be connected to each other through wires or wirelessly. For example, the Internet can be used as a communication network for connecting information processing device 6 and data management device 1.

Data management device 1 mainly serves to manage the analysis data acquired by the plurality of analyzers 4. Analysis data from each analyzer 4 is input to data management device 1. Information related to generation of the material (hereinafter also referred to as “material information”) is further input to data management device 1.

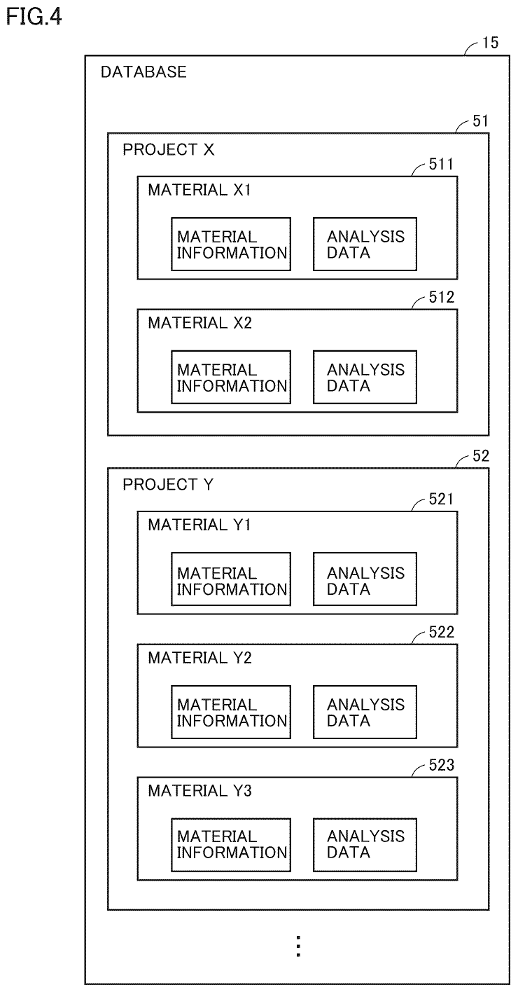

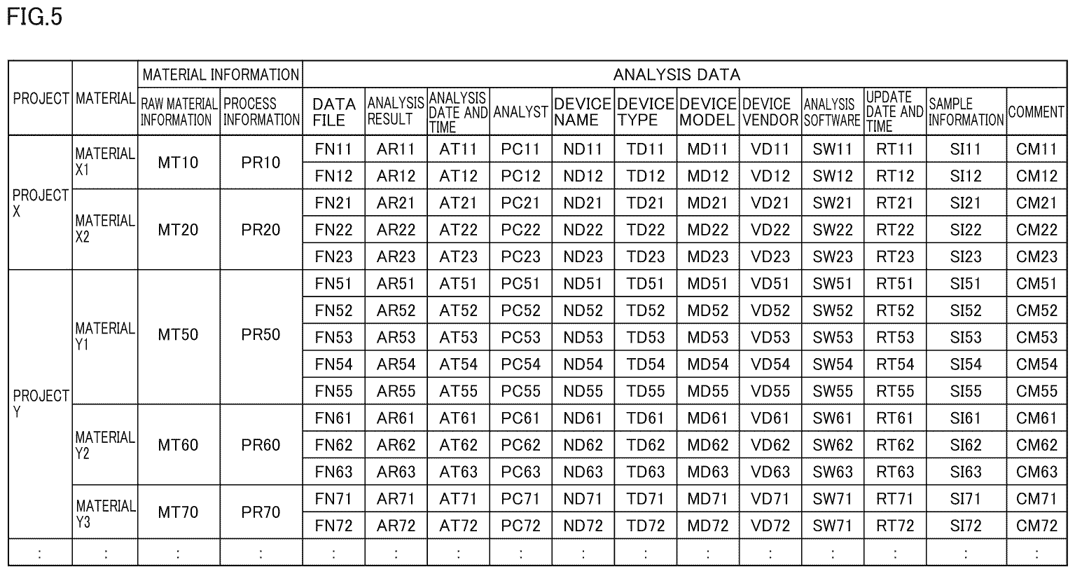

One or more projects are defined in data management device 1 as described later with reference to FIGS. 4, 5, and the like. The project is a representation freely defined by a user and represents, for example, a research or development theme. In data management device 1, information about one or more materials is registered in association with each project. Further, in data management device 1, one or more pieces of material information and one or more pieces of analysis data are registered in association with each material.

### [Hardware Configuration Example of Data Management System]

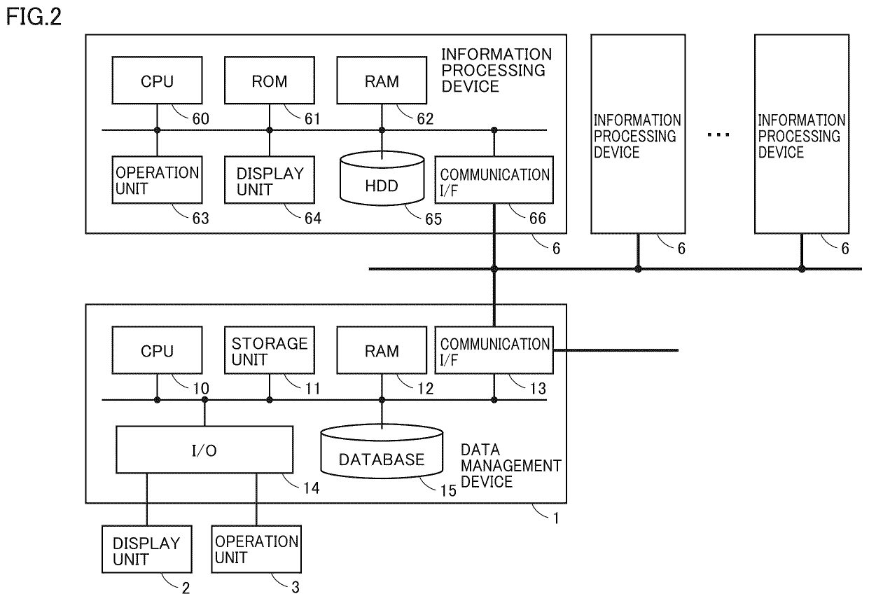

FIG. 2 is a diagram schematically showing a hardware configuration example of information processing device 6 and data management device 1. Referring to FIG. 2, hardware configurations of information processing device 6 and data management device 1 will be hereinafter described.

### (Hardware Configuration of Information Processing Device)

Information processing device 6 includes a central processing unit (CPU) 60 for controlling the entire analyzer 4, a read only memory (ROM) 61, a random access memory (RAM) 62, and a hard disk drive (HDD) 65. ROM 61, RAM 62, and HDD 65 constitute a storage unit that stores programs and data.

ROM 61 non-temporarily stores a program executed by CPU 60. RAM 62 temporarily stores data used during execution of the program in CPU 60. RAM 62 functions as a temporary data memory used as a work area. HDD 65 is a nonvolatile storage device and stores information (for example, a data file for each sample) generated by information processing device 6. A semiconductor storage device such as a flash memory may be employed in addition to or instead of HDD 65.

Information processing device 6 further includes a communication interface (I/F) 66, an operation unit 63, and a display unit 64. Communication I/F 66 is an interface through which information processing device 6 communicates with device main body 5 and external devices including data management device 1.

Operation unit 63 receives an input (for example, an instruction to information processing device 6) from a user (for example, an analyst). Operation unit 63 includes a keyboard, a mouse, a touch panel integrated with a display screen of display unit 64, and the like, and receives the measurement condition, the identification information, and the like of the sample.

When the measurement condition is set, display unit 64 can display, for example, an input screen of the measurement condition, the identification information of the sample, and the like. During the measurement, display unit 64 can display the measurement data detected by device main body 5 and the result of data analysis by information processing device 6.

The process in analyzer 4 is implemented by each hardware and software executed by CPU 60. Such software may be stored in advance in ROM 61 or HDD 65. Further, software may be stored in a storage medium (not shown) and distributed as a program product. Then, the software is read from HDD 65 by CPU 60 and stored in RAM 62 in a format executable by CPU 60. CPU 60 executes this program.

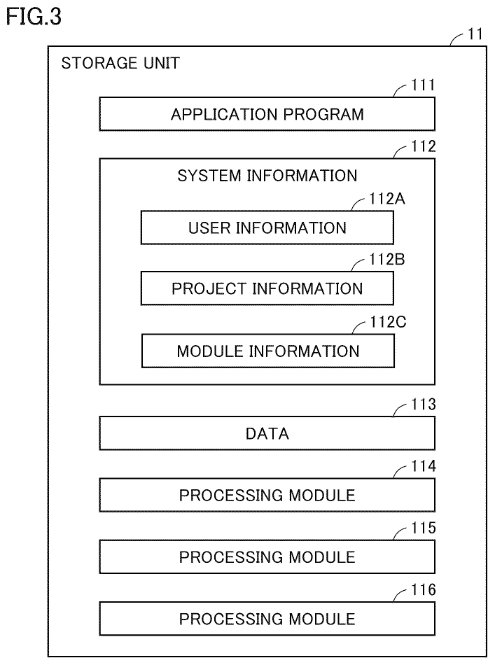

In analyzer 4, CPU 60 executes software to generate analysis data. The type of software used to generate the analysis data may be different among the plurality of analyzers 4. As will be described later with reference to FIG. 3, data management device 1 includes a plurality of types of processing modules (processing modules 114, 115, and 116). When processing the analysis data, data management device 1 selects a processing module corresponding to the type of software used to generate the analysis data from among the plurality of types of processing modules, and then uses the selected processing module for processing the analysis data.

### (Hardware Configuration of Data Management Device)

Data management device 1 includes a CPU 10 for controlling the entire device, a storage unit 11, a RAM 12, and a database 15.

Storage unit 11 non-temporarily stores a program executed by CPU 10. RAM 12 temporarily stores data used during execution of the program in CPU 10. RAM 12 functions as a temporary data memory used as a work area. Database 15, which is a non-volatile storage device, stores data exchanged between data management device 1 and the plurality of analyzers 4, data input from the outside of data management device 1, and data generated in data management device 1. An example of the structure of the data stored in database 15 will be described later with reference to FIG. 4. In the example in FIG. 2, storage unit 11 and database 15 constitute an example of a “memory”.

Data management device 1 further includes a communication I/F 13 and an input/output (I/O) interface 14. Communication I/F 13 serves as an interface through which data management device 1 communicates with external devices including information processing device 6.

I/O interface 14 serves as an interface for input to data management device 1 or output from data management device 1. I/O interface 14 is connected to a display unit 2 and an operation unit 3. Display unit 2 displays various screens including results of computation by CPU 10. Display unit 2 is implemented, for example, by a display device. Operation unit 3 receives an input including a user's instruction. Operation unit 3 is implemented, for example, by a keyboard and/or a mouse.

Data management device 1 incorporates a database. The database is a storage unit for storing: data exchanged between data management device 1 and the plurality of analyzers 4; data input from the outside of data management device 1; and data generated in data management device 1. For each material, data management device 1 stores the data file in the database in association with the material information and the physical property data of the material. In the example in FIG. 2, database 15 is incorporated in data management device 1. In data management system 100, database 15 may be configured separately from data management device 1. In this case, CPU 10 communicates with database 15 for managing the data stored in database 15.

### [Data Configuration of Storage Unit in Data Management Device]

FIG. 3 is a diagram showing an example of a data structure of storage unit 11 in data management device 1.

In the example in FIG. 3, storage unit 11 stores an application program 111, system information 112, data 113, and processing modules 114, 115, and 116. Application program 111 is executed for managing various pieces of data in data management system 100, and may be hereinafter referred to as a “management application”.

System information 112 includes data used to manage data management system 100. More specifically, system information 112 includes user information 112A, project information 112B, and module information 112C. User information 112A includes information about each user who uses data management system 100. The information about the user includes, for example, a user name and the user's contact information (an e-mail address, an account name on a social networking service, and the like).

Project information 112B includes information about each project defined in data management system 100 (a project name, information specifying a constituent user of a project, and the like). Module information 112C includes information that associates the type of analysis data with the type of the processing module to be used for processing the analysis data. In one implementation, module information 112C associates a character string (for example, an extension) included in a file name of the data file with the type of the processing module.

Data 113 includes various pieces of data used by CPU 10 for executing the program.

Each of processing modules 114, 115, and 116 is a program module and used to process the analysis data (for example, to interpret the analysis data and display the information meant by the analysis data). Processing modules 114, 115, and 116 correspond to different types of analysis software used in the analyzer. For example, processing module 114 is used for processing the analysis data generated by analysis software used in a certain model of LC-MS. Processing module 115 is used for processing the analysis data generated by analysis software used in a certain model of GC-MS. Processing module 116 is used for processing analysis data generated by analysis software used in a certain type of SEM.

In data management device 1, the management application may be executed by the operation of operation unit 3, or may be executed in response to the operation of an external terminal. In other words, in data management system 100, the management application may function as a web application. The user may access data management device 1 from the terminal of each user and cause data management device 1 to execute the management application while operating the terminal of each user.

### [Data Configuration of Database of Data Management Device]

FIG. 4 is a diagram showing an example of a data structure of database 15 in data management device 1.

As shown in FIG. 4, database 15 includes folders each storing configuration information of each project. A folder 51 represents a folder storing configuration information of a project referred to as a “project X”, and a folder 52 represents a folder storing configuration information of a project referred to as a “project Y”.

Folder 51 stores material information and analysis data about each of two materials (materials X1 and X2) registered in database 15 so as to belong to project X. A folder 511 represents an area of the information related to material X1, and a folder 512 represents an area of the information related to material X2.

In the present specification, the state in which one or more materials are managed under a certain project is represented by an expression that one or more materials “belong” to the project. One or more projects are defined in data management system 100, and one or more materials are managed under each project. For example, one or more materials produced and/or obtained for a project for developing a new product are managed under the project. In this case, the one or more materials belong to this project.

Folder 52 stores material information and analysis data about each of three materials (materials Y1, Y2, and Y3) registered in database 15 so as to belong to project Y. A folder 521 represents an area of the information related to material Y1, a folder 522 represents an area of the information related to material Y2, and a folder 523 represents an area of the information related to material Y3.

Note that the number of projects and the number of materials belonging to each project in database 15 shown in FIG. 4 are merely illustrative examples. The number of projects and the number of materials belonging to each project in database 15 are not particularly limited.

### [Specific Example of Configuration Information of Project]

FIG. 5 is a diagram showing a specific example of configuration information of each of projects. In the example in FIG. 5, materials X1 and X2 belong to project X, as in the example in FIG. 4. Materials Y1, Y2, and Y3 belong to project Y. Materials X1, X2, Y1, Y2, and Y3 represent the names of the respective materials.

The material information of each material includes raw material information and process information. For example, the raw material information of material X1 is indicated as raw material information MT10, and the process information of material X1 is indicated as process information PR10. In one implementation, the raw material information defines the substance (raw material) and the amount thereof used to produce the material. Further, the process information defines the condition for producing the material. For example, when the material is a three-way catalyst, the raw material information includes a blending amount (g) of platinum (Pt) and a blending amount (g) of palladium (Pd), and the process information includes stirring time (min) and a firing temperature (° C.). Note that the material information may further include information indicating the content of a main agent, the content of a hardening agent, how to mix the raw materials, and/or whether or not the mixed raw materials are hardened in five minutes.

In the example in FIG. 5, the raw material information indicated by each piece of raw material information MT10, MT20, MT50, MT60, and MT70 is registered for each of respective materials X1, X2, Y1, Y2, and Y3. Further, the process information indicated by each piece of process information PR10, PR20, PR50, PR60, and PR70 is registered for each of respective materials X1, X2, Y1, Y2, and Y3.

In the example in FIG. 5, twelve items (a data file, an analysis result, analysis date and time, an analyst, a device name, a device type, a device model, a device vendor, analysis software, update date and time, sample information, and a comment) are defined for the analysis data.

The “data file” indicates a file name of the analysis data.

The “analysis result” indicates a measurement result and an analysis result obtained in the analyzer. For example, when the analysis data is generated in LC-MS, the analysis result includes a count value at each m/z as an example of the measurement result, and includes a peak area as an example of the analysis result.

The “analysis date and time” indicates the date and time at which the measurement based on the analysis data was performed in the analyzer.

The “analyst” indicates a person who has performed the measurement based on the analysis data. In one implementation, the analyzer requests a user to log in when performing the measurement. The logged-in user is specified as the analyst.

The “device name” indicates a name allocated in data management system 100 to the analyzer that has performed the measurement based on the analysis data.

The “device type” indicates the type of the analyzer that has performed the measurement based on the analysis data. The type of the analyzer is, for example, LC, GC, LC-MS, GC-MS, Py-GC/MS, SEM, TEM, EDX, WDX, NMR, FT-IR, LC-PDA, LC/MS/MS, GC/MS/MS, LC/MS-IT-TOF, a near infrared spectrometer, a tensile tester, a compression tester, AES, AAS/FLAAS, ICP-MS, an organic element analyzer, GDMS, a particle composition analyzer, TN, or NC.

The “device model” indicates the model (model number) of the analyzer that has performed the measurement based on the analysis data.

The “device vendor” indicates a manufacturer and/or a seller of the analyzer that has performed the measurement based on the analysis data.

The “analysis software” indicates the software used to generate analysis data in the analyzer that has performed the measurement based on the analysis data.

The “update date and time” indicates the date and time at which the analysis data has been updated in data management device 1.

The “sample information” indicates the information of a sample subjected to the measurement based on the analysis data. In one implementation, the sample information includes the name and/or the ID allocated to the sample in data management system 100, the amount of the sample subjected to the measurement, and/or the dilution rate of an undiluted solution in the sample.

The “comment” indicates a comment added to the analysis data.

Note that the number and the type of each item whose values are registered in the analysis data are not limited to those shown in FIG. 5. Other types of items that may be included may be: other types of items (stirring time, an amount of polystyrene in the sample, a particle size of the sample, and/or the type of a tester), ISTD group numbers (numbers of groups based on the applied internal standard method), and/or feature amounts (a mass, a peak area, a peak height, a ratio of an area to a height, a peak number, standard hold time, a waveform processing mode, hold time, a hold index, a concentration unit, peak start time, peak end time, a type of a calibration curve, an intercept of a calibration curve, a first-order coefficient of a calibration curve, a second-order coefficient of a calibration curve, and/or a third-order coefficient of a calibration curve) regarding specific compounds (diisobutyl phthalate (DIBP), dibutyl phthalate (DBP), and the like). Further, in the case where the analysis data is acquired by SEM, the types of items include a contrast, brightness, an acceleration voltage, a magnification, a scan mode, a standard scan speed, a signal name, stage coordinates, date of recording, time of recording, a recorder who made recordings, a device name, a label, offset X/Y, an image size X/Y before being cut out, a detector name, an electron gun voltage in a gentle beam (GB) mode, a sample voltage in a GB mode, a working distance, a column mode, a depth of focus, an objective lens aperture, a cathode luminescence (CL) course value, a CL fine value, a probe number, an irradiation current fine value, a scan rotation angle, the number of integrations, an emission current, a probe current, the degree of vacuum of a Penning gauge, the degree of vacuum at a low voltage (LV), and/or a vacuum mode.

In the example in FIG. 5, the information about one analysis data is shown in each row. For example, the analysis data specified by a data file FN11 is analysis data generated based on the measurement of a sample that has been sampled from material X1. The analysis data further includes an analysis result AR11, analysis date and time AT11, an analyst PC11, a device name ND11, a device type TD11, a device model MD11, a device vendor VD11, analysis software SW11, update date and time RT11, sample information SI11, and a comment CM11.

In the example in FIG. 5, for material X1, the analysis data specified by data file FN11 and the analysis data specified by a data file FN12 are registered. For material X2, the analysis data specified by a data file FN21, the analysis data specified by a data file FN22, and the analysis data specified by a data file FN23 are registered. For material Y1, the analysis data specified by a data file FN51, the analysis data specified by a data file FN52, the analysis data specified by a data file FN53, the analysis data specified by a data file FN54, and the analysis data specified by a data file FN55 are registered. For material Y2, the analysis data specified by a data file FN61, the analysis data specified by a data file FN62, and the analysis data specified by a data file FN63 are registered. For material Y3, the analysis data specified by a data file FN71 and the analysis data specified by a data file FN72 are registered.

### Screen Display Example (1)

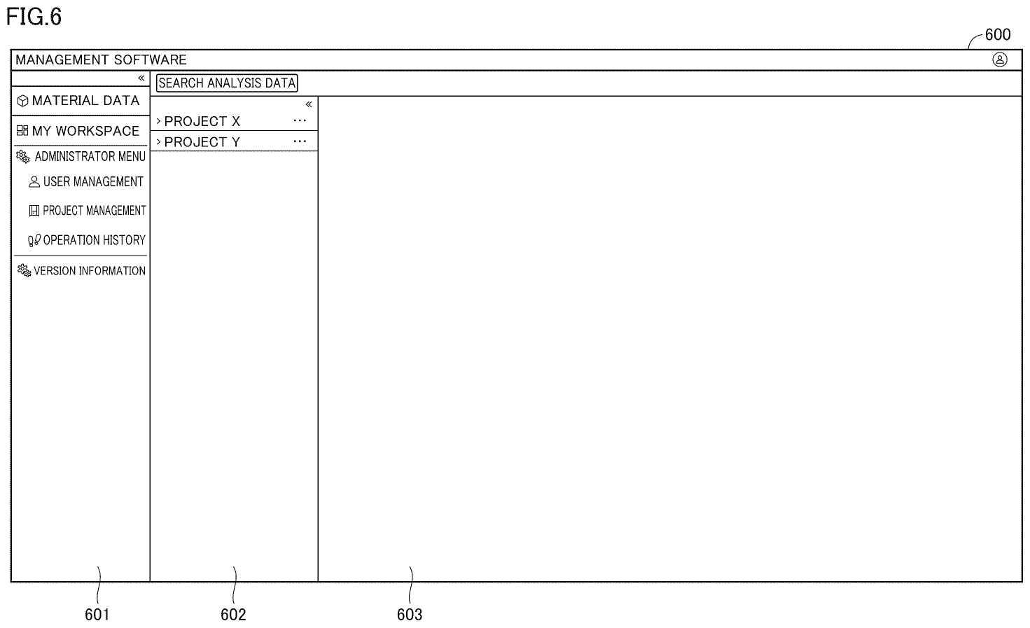

FIG. 6 is a diagram showing an example of a display screen of the management application. A screen 600 in FIG. 6 includes fields 601, 602, and 603.

Field 601 displays a menu (material data, My workspace, an administrator menu, user management, project management, an operation history, version information) selected in the management application.

Field 602 displays a list of projects registered in the management application (database 15). In the example in FIG. 6, two projects (projects X and Y) are displayed.

Field 603 displays information related to an element as a target to be worked (for example, a project, a material, material information, analysis data, and the like).

In the example in FIG. 6, since the element as a target to be worked is not displayed, field 603 shows nothing.

### Screen Display Example (2)

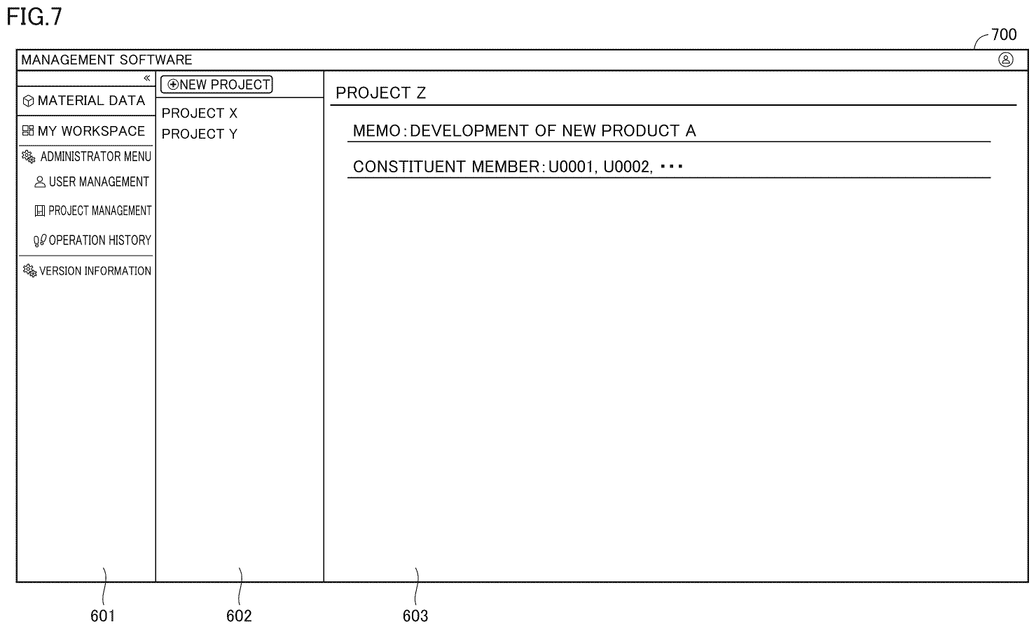

FIG. 7 is a diagram showing another example of the display screen of the management application. A screen 700 in FIG. 7 is displayed when an operation to register a new project in data management system 100 is performed in the management application.

Field 603 in FIG. 7 displays the information input by the user to be registered as a new project. More specifically, field 603 displays a project name (a project Z), a memo related to the project (development of a new product A), and constituent members (U0001, U0002, . . . ) of the project. Any character string may be registered as a project name and a memo. Each of “U0001” and “U0002” represents an ID of the user registered in data management system 100.

### Screen Display Example (3)

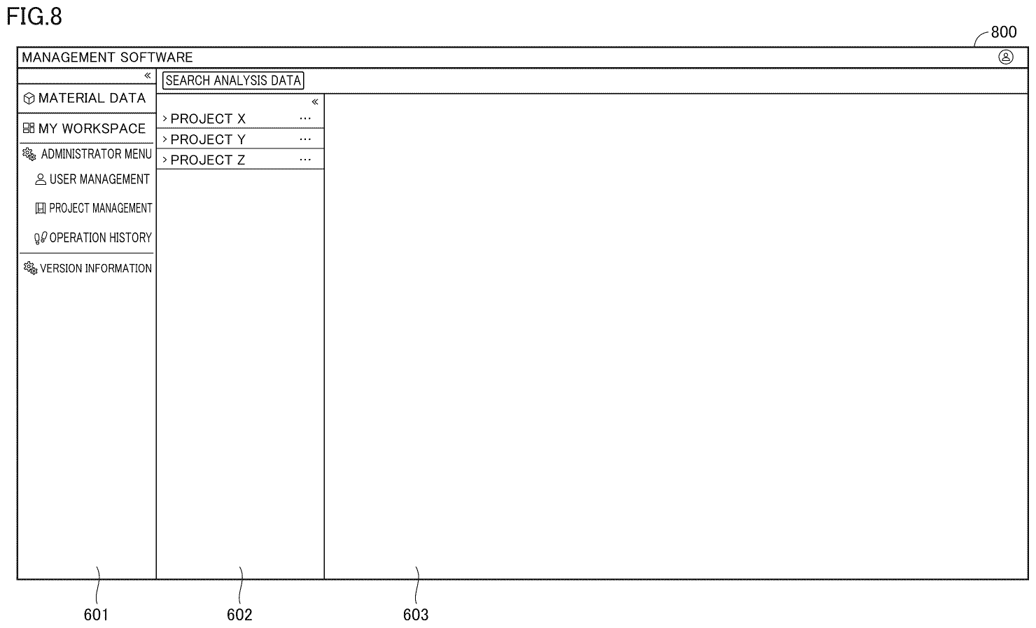

FIG. 8 is a diagram showing still another example of the display screen of the management application. A screen 800 in FIG. 8 displays the screen in FIG. 7 in which “project Z” is newly added to the screen in FIG. 6. Field 602 on screen 800 further displays “project Z” as compared with field 602 on screen 600.

### Screen Display Example (4)

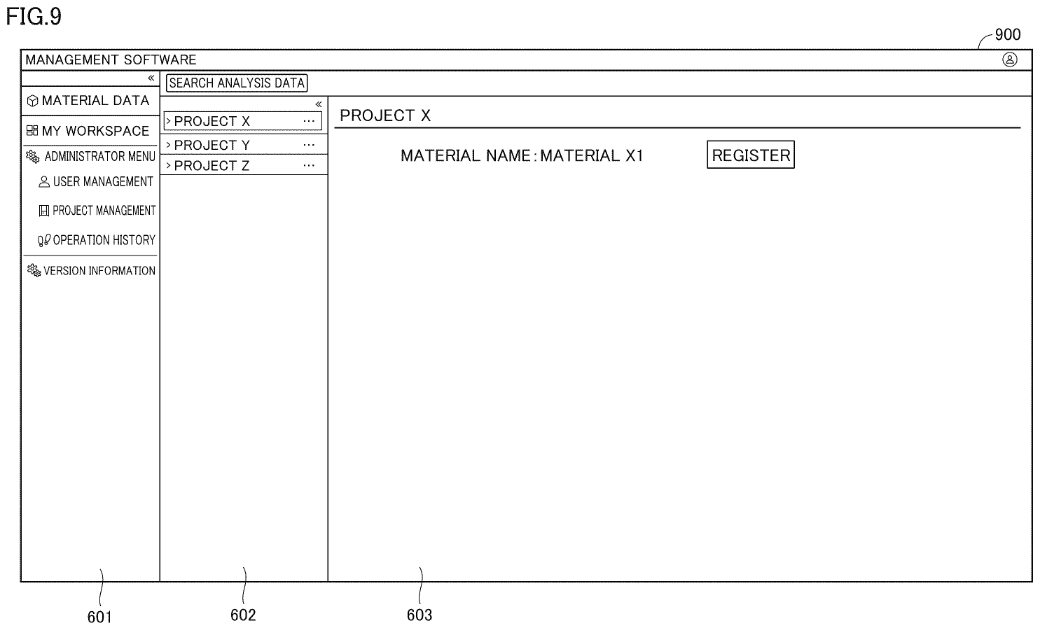

FIG. 9 is a diagram showing still another example of the display screen of the management application. A screen 900 in FIG. 9 is for registering a material belonging to the project registered in the management application.

In the example in FIG. 9, project X is selected as a project for which the material is registered. Thereby, in field 602, “project X” is highlighted among the three projects (projects X, Y, and Z) registered in the management application.

In field 603, the material intended to be registered so as to belong to project X, i.e., “material X1”, is displayed together with a register button. When the user inputs (the name of) the material (i.e., material X1) to be registered to field 603, the management application displays a register button in association with the material. When the register button is operated, the management application registers the material in database 15 in association with the project to be processed (“project X” in the example in FIG. 9). More specifically, the management application generates, in database 15, a folder (a storage area) for the newly registered material in the folder (the storage area) corresponding to the project to be processed.

When accepting registration of a new material for the project to be processed, the management application may cause field 603 to display the information about the already registered material. Further, the management application may simultaneously accept registrations of a plurality of materials in field 603.

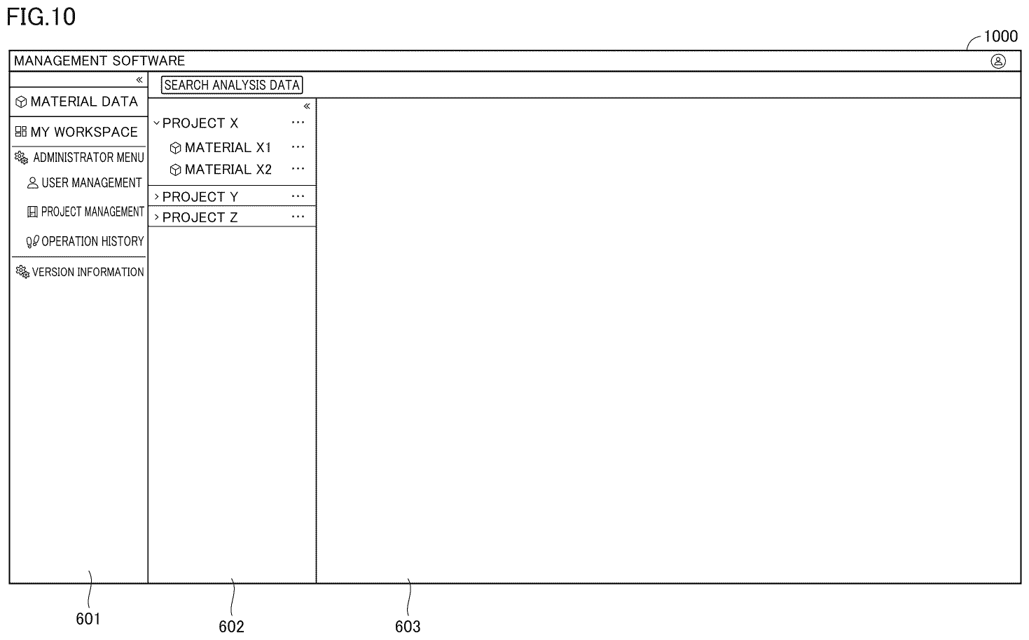

FIG. 10 is a diagram showing still another example of the display screen of the management application. When a material belonging to a certain project is registered, the management application displays the information about the registered material in association with this certain project. In the example in FIG. 10, in response to registration of materials X1 and X2 as the materials belonging to project X, materials X1 and X2 are displayed in field 602 in association with project X. In one implementation, in field 602, each time a click operation is performed on the name of a project, the display of the material belonging to this project is switched on and off.

### Screen Display Example (5)

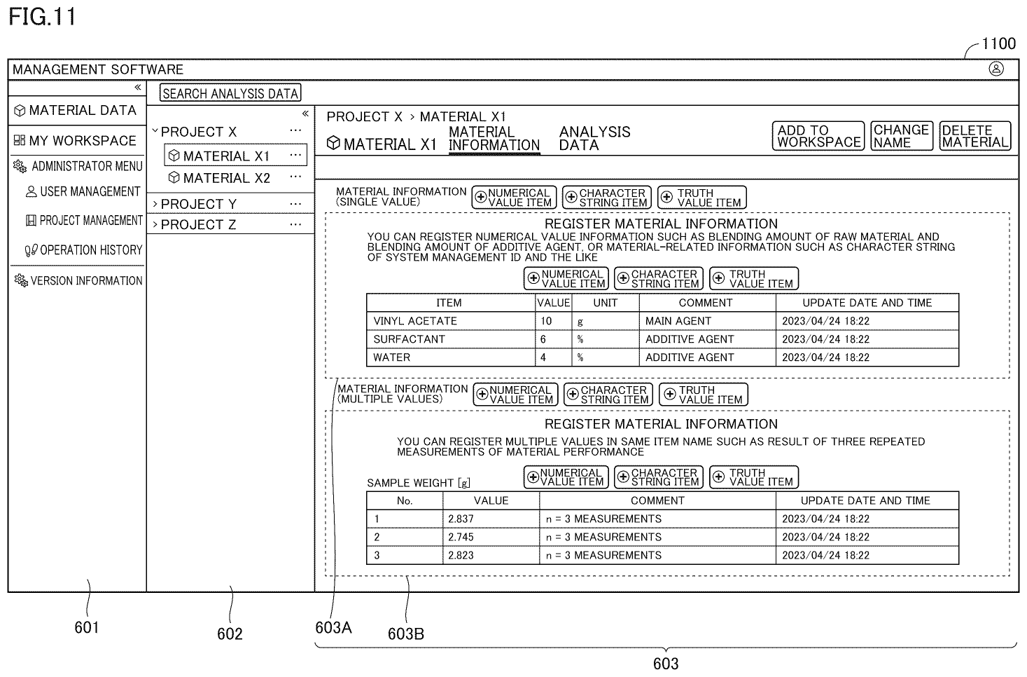

FIG. 11 is a diagram showing still another example of the display screen of the management application. A screen 1100 in FIG. 11 is for registering material information of a material.

Field 602 in FIG. 11 shows information for selecting a project and a material to be processed. In the example in FIG. 11, material X1 belonging to project X is selected as a target to be processed. In one implementation, when the “material information” tab is selected from among two tabs (“material information” and “analysis data”) in the upper section of field 603, the management application is brought into a state in which it accepts registration of the material information.

Frames 603A and 603B of field 603 show information that is input as material information. More specifically, frame 603A shows raw materials (vinyl acetate, a surfactant, and water) of material X1 and blending amounts of the respective raw materials (“10 g” of vinyl acetate, “6%” of the surfactant, and “4%” of water). Frame 603B shows the sample weight at the time when the raw materials of material X1 are weighed.

In response to execution of the operation by the user to register the input information in the state in which the information is input into field 603, the management application registers the input information as material information in database 15.

### Screen Display Example (6)

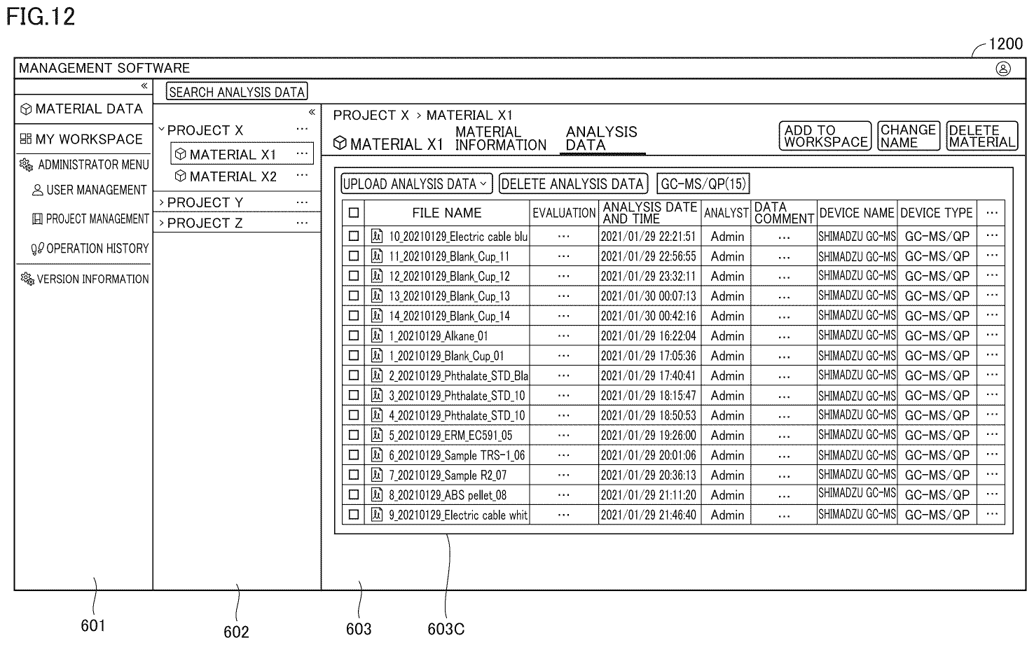

FIG. 12 is a diagram showing still another example of the display screen of the management application. A screen 1200 in FIG. 12 is for registering analysis data of a material. Registering the analysis data in database 15 from the outside of data management device 1 may be referred to as uploading of the analysis data.

Field 602 in FIG. 12 shows information for selecting a project and a material to be processed. In the example in FIG. 12, material X1 belonging to project X is selected as a target to be processed. In the example in FIG. 12, material X1 constitutes an example of the “first material”, and screen 1200 for registering the analysis data of material X1 constitutes an example of the “registration screen” related to the first material.

In one implementation, when the “analysis data” tab is selected from among two tabs (“material information” and “analysis data”) in the upper section of field 603, the management application is brought into a state in which it accepts registration of the analysis data.

A table 603C in field 603 shows a list of the analysis data stored in information processing device 6 accessible by data management device 1. More specifically, in order to display table 603C, data management device 1 that executes the management application accesses one or more information processing devices 6 in data management system 100, and acquires the information of the analysis data stored in each information processing device 6. Then, based on the acquired information, data management device 1 displays a list of the analysis data as table 603C. The list shows values of one or more items (an analysis data name (a data file), analysis date and time, an analyst, . . . ) of each piece of analysis data as described with reference to FIG. 5. In the example in FIG. 12, “evaluation” is further included as an item of the analysis data. Table 603C shows a check box associated with each piece of analysis data.

The user marks the check box of the analysis data to be registered in association with the material to be processed, and then performs an upload operation. In response to execution of the upload operation, the management application registers the marked analysis data in database 15 as analysis data associated with the material to be processed.

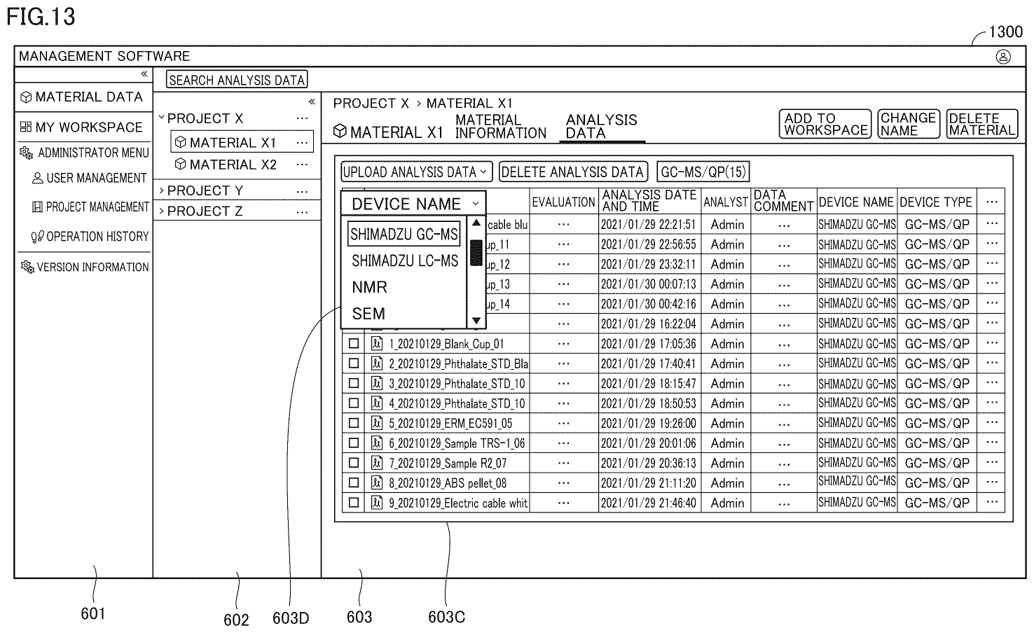

FIG. 13 is a diagram showing still another example of the display screen of the management application. A screen 1300 in FIG. 13 constitutes a modification of screen 1200 in FIG. 12.

As shown in FIG. 13, the management application further displays a pull-down menu 603D on screen 1300. Pull-down menu 603D is displayed for selecting the device name corresponding to the analysis data displayed in table 603C. The user selects the device name in pull-down menu 603D. The management application selects analysis data associated with the selected device name from among the respective pieces of analysis data stored in one or more information processing devices 6 in data management system 100. Then, the management application displays a list of the selected analysis data in table 603C.

Note that the item selected in the pull-down menu is not limited to the device name. Other items such as a device type may also be selected. In the case where a large number of pieces of analysis data are stored in one or more information processing devices 6 of data management system 100 by selection using such a pull-down menu, data management device 1 can narrow down the analysis data displayed in table 603C as desired by the user.

### Screen Display Example (7)

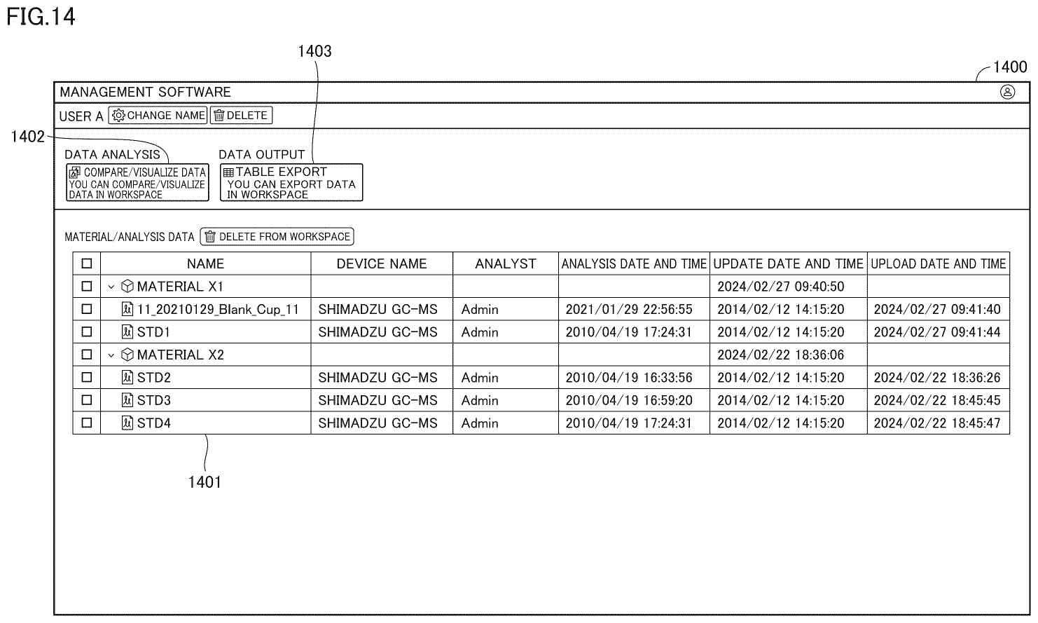

FIG. 14 is a diagram showing still another example of the display screen of the management application. A screen 1400 in FIG. 14 is for displaying, for a certain project, information of the analysis data registered in association with each of one or more materials belonging to this certain project.

In the example in FIG. 14, “project X” is selected as a project to be processed. Screen 1400 includes a table 1401. Table 1401 shows information of the analysis data registered in database 15 in association with each of two types of materials (materials X1 and X2) belonging to project X. The “upload date and time” of each piece of analysis data in FIG. 14 means the date and time at which the analysis data was registered in database 15.

When the user clicks the file name (“11_20210129_Blank_Cup_11”, “STD1”, “STD2”, “STD3”, or “STD4”) of the analysis file displayed in table 1401, the management application displays the analysis data specified by the clicked file name.

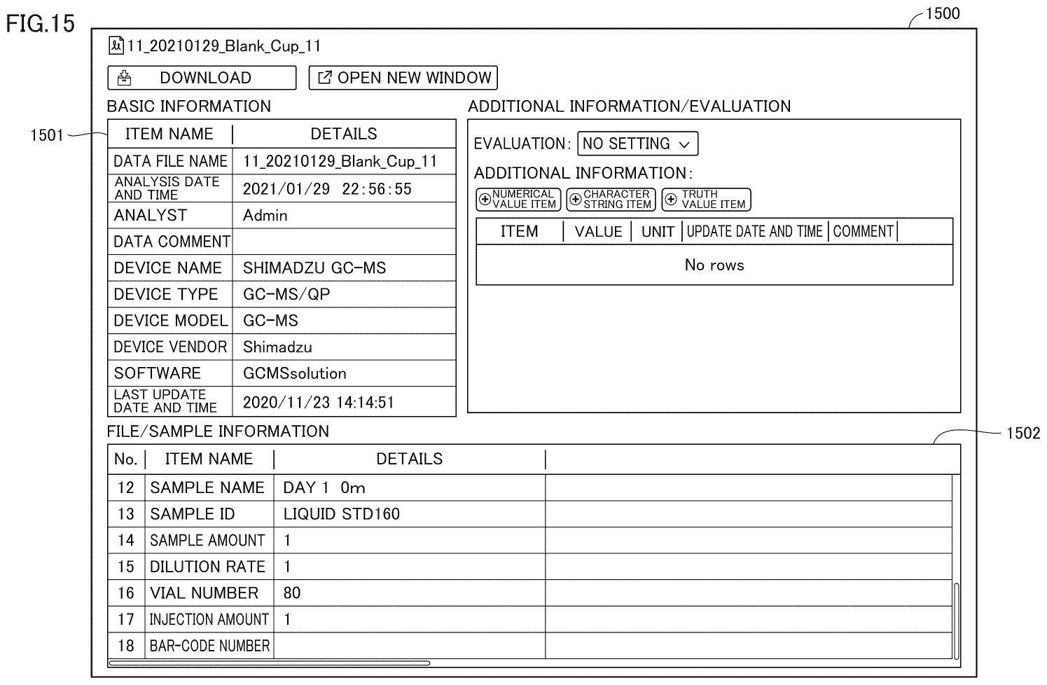

FIG. 15 is a diagram showing still another example of the display screen of the management application. A screen 1500 in FIG. 15 is for displaying analysis data.

In the example in FIG. 15, screen 1500 includes a field 1501 showing basic information and a field 1502 showing file/sample information. The basic information includes a measurement result, a measurement condition, and an analysis result for the analysis data. The file/sample information includes identification information of the sample in the analysis data. On screen 1500, the user can edit the analysis data. More specifically, the management application receives, in each field of screen 1500, a user input for adding or changing data and an input for confirming the user input. Upon receipt of the input of confirmation, the management application updates the analysis data in database 15 in accordance with the user input.

Referring back to FIG. 14, on screen 1400, table 1401 includes check boxes associated with the respective pieces of analysis data, and also includes buttons 1402 and 1403.

When the user operates button 1402 in the state in which the check boxes of one or more pieces of analysis data are marked in table 1401, the management application displays the one or more pieces of marked analysis data in a comparable manner.

When the user operates button 1403 in the state in which the check boxes of the one or more pieces of analysis data are marked, the management application collectively exports the one or more pieces of marked analysis data to an external device. Thereby, the management application can collectively export a plurality of pieces of analysis data each having a plurality of items to the external device. Screen 1400 including table 1401 constitutes an example of an “analysis data selection screen”.

In the present embodiment, the analysis data is managed as tabular data as described with reference to FIG. 5. The external device may process the analysis data acquired from data management device 1 with the use of statistical analysis software. When the analysis data exported from data management device 1 is exported in a tabular form to the external device, the analysis data can be exported in a format that is easily readable by the external device.

### Screen Display Example (8)

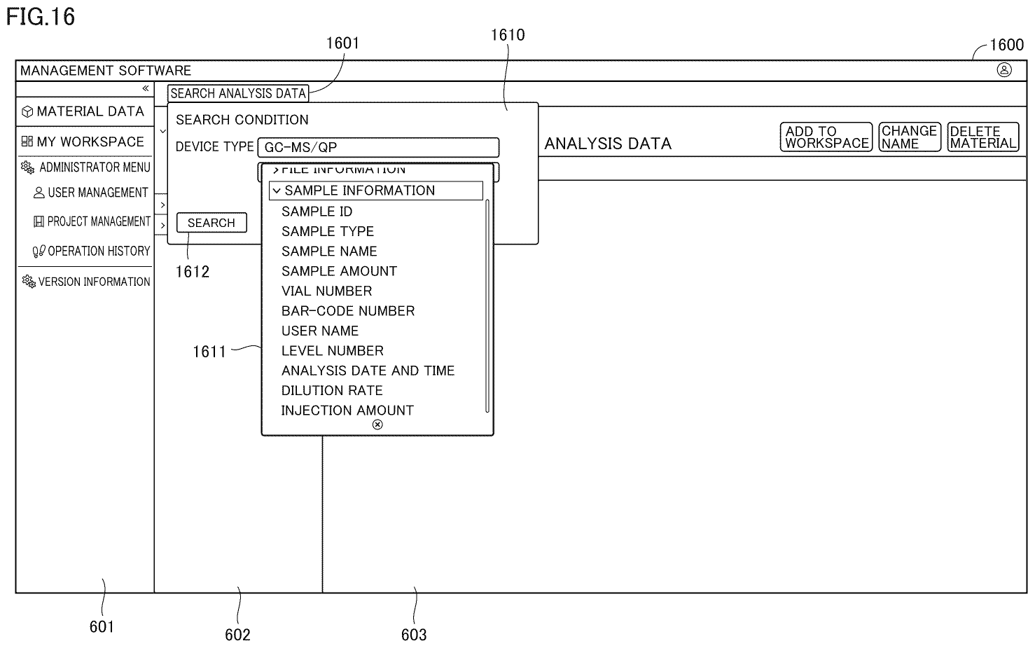

FIG. 16 is a diagram showing still another example of the display screen of the management application. A screen 1600 in FIG. 16 is for inputting the search condition for the analysis data registered in database 15. Screen 1600 includes a search button 1601. When search button 1601 is operated, the management application displays a pop-up window 1610 through which the search condition is input.

In the example in FIG. 16, pop-up window 1610 shows an item “device type” and a value “GC-MS/QS” of this item. When a given operation is performed on pop-up window 1610, the management application displays a list window 1611. List window 1611 displays a list of items designated as a search condition. List window 1611 displays items included in the analysis data.

More specifically, list window 1611 in FIG. 16 shows specific items included in the sample information obtained when the analysis data is acquired by LC-MS.

When the user selects an item, the management application displays the selected item on pop-up window 1610, and displays a frame next to the item such that a value as a search condition of the selected item is shown in the frame. The user inputs a value to be set as a search condition into the frame. In this way, the user sets the search condition on pop-up window 1610.

Pop-up window 1610 includes a search button 1612. When search button 1612 is operated, the management application searches the analysis data in database 15 according to the search condition input into pop-up window 1610.

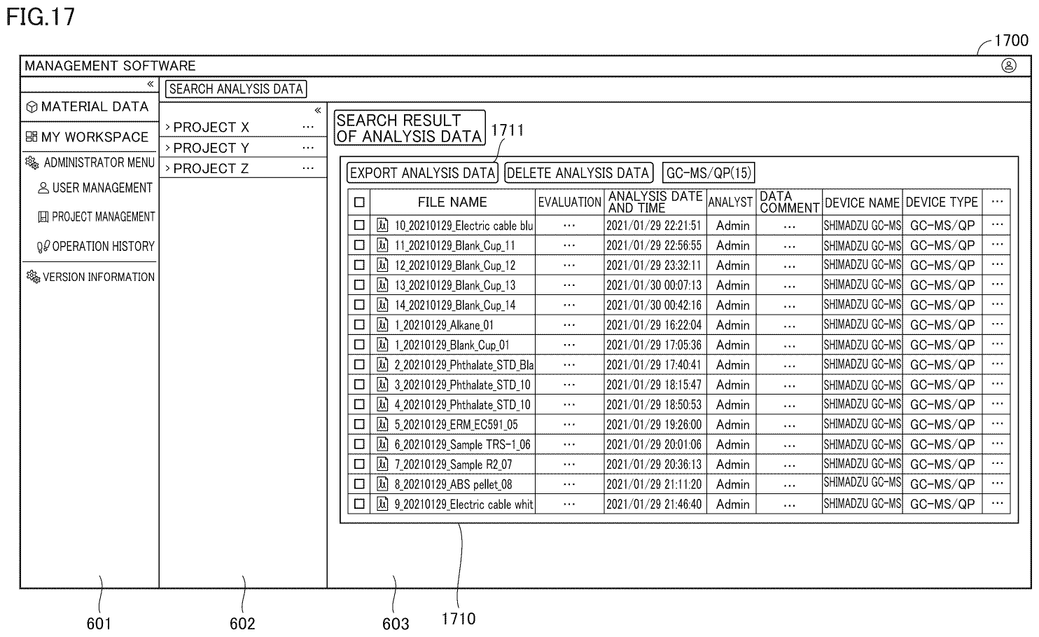

FIG. 17 is a diagram showing still another example of the display screen of the management application. A screen 1700 in FIG. 17 displays a search result of the analysis data. Screen 1700 includes a field 1710. Field 1710 includes a list showing the information of the analysis data acquired as the search result. The list includes a check box and a file name (the name of the “data file” in FIG. 5) of each piece of analysis data. When a file name of certain analysis data is clicked, the management application displays the clicked analysis data, for example, as described with reference to FIG. 15.

Field 1710 includes a button 1711. When button 1711 is operated in the state in which one or more check boxes are marked in the list in field 1710, the management application performs control for export. More specifically, after receiving the designation of the export destination of the one or more pieces of marked analysis data, the management application exports the one or more pieces of marked analysis data to the export destination. In the example described with reference to FIGS. 16 and 17, the user can set the search condition for the analysis data, check each of the one or more pieces of analysis data obtained as the search result, and instruct the management application to collectively export the one or more pieces of analysis data obtained as the search result to an external device. Screen 1700 including field 1710 constitutes an example of the “analysis data selection screen”.

### [Process Flow]

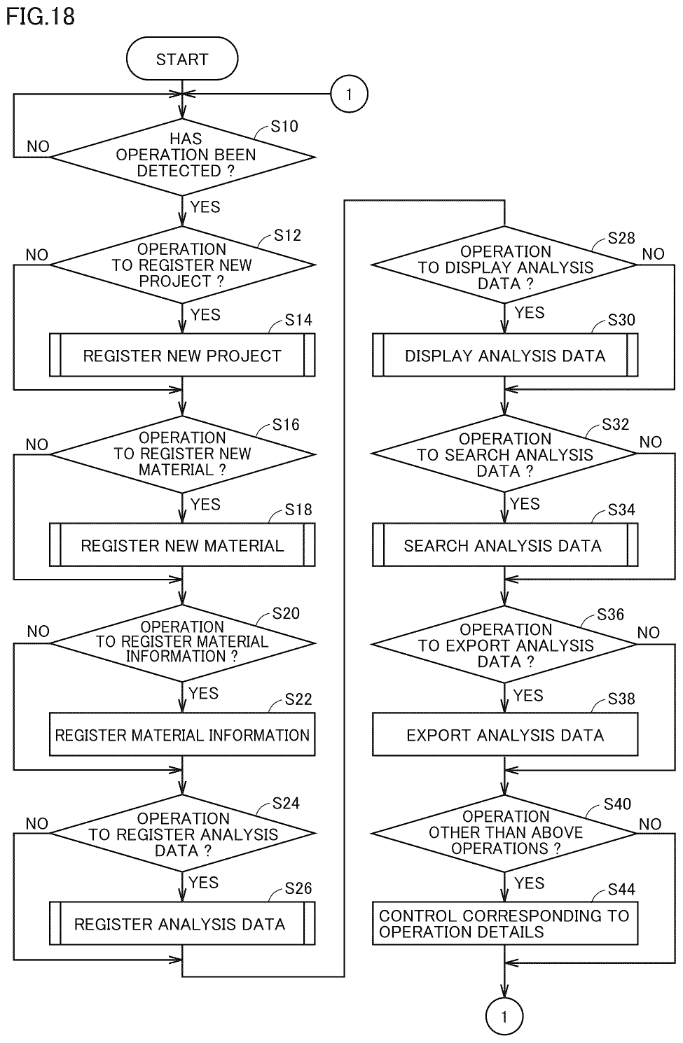

FIG. 18 is a flowchart of a process performed by execution of the management application by data management device 1. In one implementation, in data management device 1, CPU 10 executes a program of the management application.

Referring to FIG. 18, in step S10, data management device 1 determines whether or not an operation in the management application has been detected. Data management device 1 suspends the control in step S10 until it is determined that the operation has been detected (NO in step S10). When it is determined that the operation has been performed (YES in step S10), data management device 1 advances the control to step S12.

In step S12, data management device 1 determines whether or not the operation detected in step S10 is an operation to register a new project. If so (YES in step S12), data management device 1 advances the control to step S14, and if not (NO in step S12), it advances the control to step S16. Note that data management device 1 displays, for example, screen 700 in FIG. 7 for registering a new project.

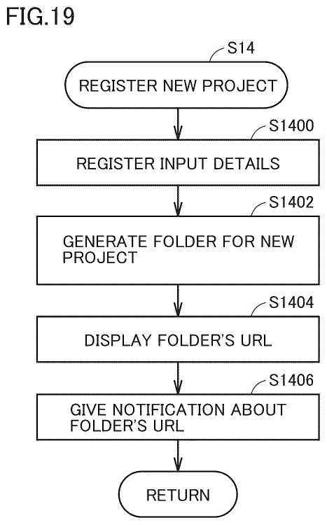

In step S14, data management device 1 performs a process of registering a new project. Then, data management device 1 advances the control to step S16. FIG. 19 is a flowchart of a subroutine of a process of registering a new project. The details of step S14 will be described with reference to FIG. 19.

In step S1400, data management device 1 registers the information input for the new project in storage unit 11 as project information 112B (FIG. 3).

In step S1402, data management device 1 generates (sets) a folder for a new project in database 15.

In step S1404, data management device 1 causes display unit 2 to show the uniform resource locator (URL) of the folder generated in step S1402.

In step S1406, data management device 1 notifies the user registered as a constituent member of the new project in step S1400 about the URL of the folder generated in step S1402. The notification may be made by transmission of e-mail or made by transmission of a message through the SNS. This allows the member of the new project to easily access the material information and the analysis data related to the project. Then, data management device 1 returns the control to FIG. 18.

Referring back to FIG. 18, in step S16, data management device 1 determines whether or not the operation detected in step S10 is an operation to register a new material. If so (YES in step S16), data management device 1 advances the control to step S18, and if not (NO in step S16), it advances the control to step S20. Note that data management device 1 displays, for example, screen 900 in FIG. 9 for registering a new material.

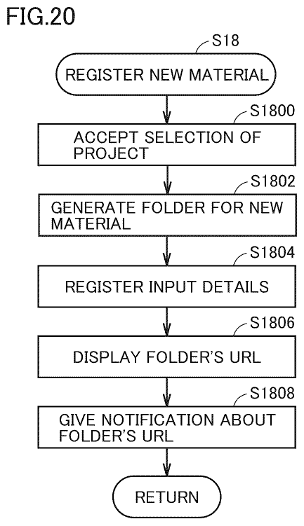

In step S18, data management device 1 performs a process of registering a new material. Then, data management device 1 advances the control to step S20. FIG. 20 is a flowchart of a subroutine of a process of registering a new material. The details of step S18 will be described with reference to FIG. 20.

In step S1800, data management device 1 accepts selection of a project to which a material to be registered belongs.

In step S1802, in database 15, data management device 1 generates a folder for the material to be registered, in the folder of the project selected in step S1800.

In step S1804, data management device 1 registers the input details (for example, material information) about the new material in the folder generated in step S1802.

In step S1806, data management device 1 causes display unit 2 to show the URL of the folder generated in step S1802.

In step S1808, data management device 1 notifies the constituent member of the project to which the new material belongs about the URL of the folder generated in step S1802. Then, data management device 1 returns the control to FIG. 18.

Referring back to FIG. 18, in step S20, data management device 1 determines whether or not the operation detected in step S10 is an operation to register material information. If so (YES in step S20), data management device 1 advances the control to step S22, and if not (NO in step S20), it advances the control to step S24. Note that data management device 1 displays, for example, screen 1100 in FIG. 11 for registering the material information.

In step S22, data management device 1 performs a process of registering the material information. More specifically, data management device 1 specifies the project and the material corresponding to the material information to be registered. Then, in database 15, data management device 1 registers the input information as the material information in the folder of the specified material of the specified project. Then, data management device 1 advances the control to step S24.

Referring back to FIG. 18, in step S24, data management device 1 determines whether or not the operation detected in step S10 is an operation to register analysis data. If so (YES in step S24), data management device 1 advances the control to step S26, and if not (NO in step S24), it advances the control to step S28. Note that data management device 1 displays, for example, screen 1200 in FIG. 12 and/or screen 1300 in FIG. 13 for registration of analysis data.

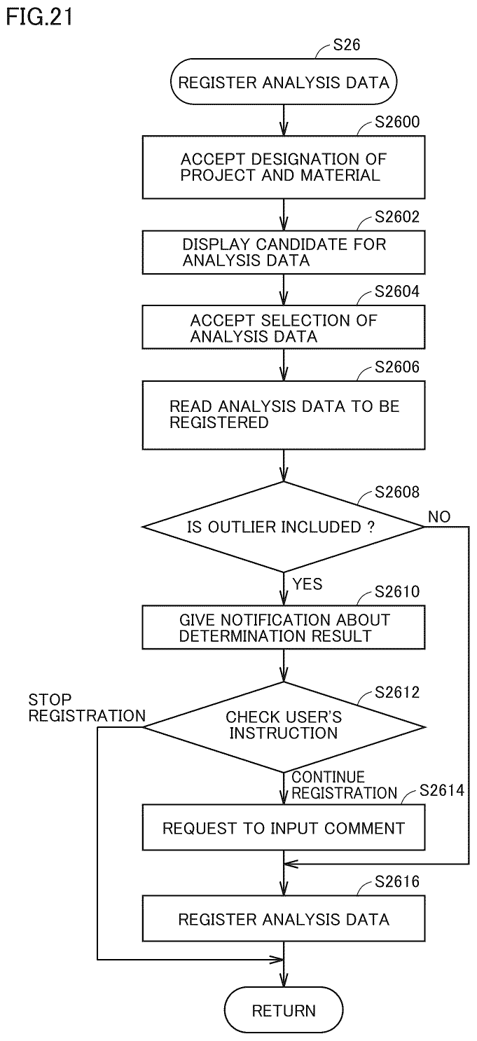

In step S26, data management device 1 performs a process of registering the analysis data. Then, data management device 1 advances the control to step S28. FIG. 21 is a flowchart of a subroutine of a process of registering analysis data. The details of step S26 will be described with reference to FIG. 21.

In step S2600, data management device 1 accepts designation of a project and a material, each of which is a target of the analysis data to be registered. This designation is implemented, for example, by selecting a project and a material in field 602 in FIG. 12.

In step S2602, data management device 1 displays a candidate for the analysis data to be registered. The candidate to be displayed may be all the analysis data stored in the device with which data management device 1 can communicate, or may be analysis data selected according to the condition specified in pull-down menu 603D in FIG. 13.

In step S2604, data management device 1 accepts selection of the analysis data to be registered. The selection is made, for example, by marking a checkbox in table 603C in FIG. 12.

In step S2606, data management device 1 reads the analysis data to be registered (the analysis data selected in step S2604) from each external device in which each piece of analysis data is stored.

In step S2608, data management device 1 determines whether or not an outlier is included in each piece of analysis data read in step S2606.

In one implementation, when determining whether or not an outlier is included, for the material corresponding to the analysis data to be registered (the analysis data as a target to be determined in step S2608 whether or not an outlier is included), the value of other analysis data already stored in database 15 is used as a reference value. In this case, whether or not an outlier is included may be determined by using a k-nearest neighbor (kNN) method or a local outlier factor (LOF) method, or may be determined by determining whether or not a difference between the value of the analysis data to be registered and an average value of other pieces of analysis data exceeds a predetermined threshold value.

In another implementation, the above-mentioned determination is made by using a reference value registered in advance in storage unit 11 for the material corresponding to the analysis data to be registered. More specifically, when the difference between the value included in the analysis data to be registered and the reference value exceeds a predetermined threshold value, the determination result indicates that an outlier is included in the analysis data.

If the determination result in step S2608 shows that “an outlier is included in the analysis data to be registered” (YES in step S2608), data management device 1 advances the control to step S2610, and if not (NO in step S2608), it advances the control to step S2616.

In step S2610, data management device 1 gives a notification about the determination result (an outlier is included in the analysis data to be registered).

In step S2612, data management device 1 checks the user's instruction about the analysis data determined as including an outlier. In one implementation, in step S2610, data management device 1 may give a notification and also inquire of the user whether or not to continue the registration of the analysis data that has been determined as including an outlier. In step S2612, data management device 1 checks the details of the user's instruction to the inquiry. If the user's instruction is to continue the registration of the analysis data, data management device 1 advances the control to step S2614, and if the user's instruction is to stop the registration of the analysis data, data management device 1 directly returns the control to FIG. 18.

In step S2614, data management device 1 requests the user to input a comment about the registration of the analysis data determined as including an outlier. Then, data management device 1 advances the control to step S2616.

In step S2616, data management device 1 registers the analysis data read in step S2606 in database 15, and returns the control to FIG. 18. When the comment is input in response to the request in step S2614, then in step S2616, data management device 1 registers the comment as a value of the item “comment” of the analysis data to be registered.

In the process in FIG. 21, steps S2606 to S2616 are performed for each piece of analysis data. In other words, when a plurality of pieces of analysis data are selected in step S2604, data management device 1 reads each of the plurality of pieces of analysis data in step S2606, and determines in step S2608 whether or not an outlier is included in each piece of analysis data. In step S2616, data management device 1 registers, directly in database 15, the analysis data determined as not including an outlier. On the other hand, for the analysis data determined as including an outlier, data management device 1 performs steps S2610 to S2616.

Referring back to FIG. 18, in step S28, data management device 1 determines whether or not the operation detected in step S10 is an operation to display analysis data. If so (YES in step S28), data management device 1 advances the control to step S30, and if not (NO in step S28), it advances the control to step S32. Data management device 1 receives an operation to display the analysis data, for example, as an operation on table 1401 in screen 1400 in FIG. 14.

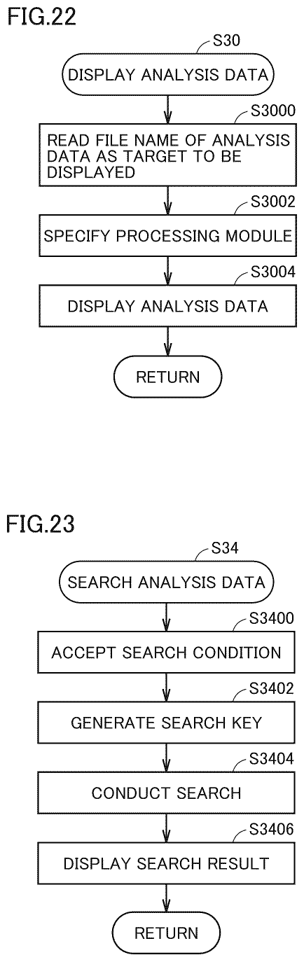

In step S30, data management device 1 performs a process of displaying the analysis data. Then, data management device 1 advances the control to step S32. FIG. 22 is a flowchart of a subroutine of a process of displaying the analysis data. The details of step S30 will be described with reference to FIG. 22.

In step S3000, data management device 1 reads a file name of the analysis data selected as a target to be displayed.

In step S3002, based on the file name read in step S3000, data management device 1 specifies a processing module to be used for displaying the analysis data selected as the target to be displayed. More specifically, data management device 1 searches the file name read in step S3000 for a character string registered in module information 112C. Then, data management device 1 specifies, in module information 112C, the type of the processing module associated with the character string included in the file name as a processing module to be used for displaying the analysis data. The character string to be searched for may be a character string that constitutes an extension of the file or may be a character string that does not constitute an extension of the file.

In step S3004, by using the processing module specified in step S3002, data management device 1 displays the analysis data to be displayed. Then, data management device 1 returns the control to FIG. 18.

When the analysis data includes a value in the item of the analysis software, data management device 1 may specify the processing module to be used for displaying the analysis data, based on this value instead of the control in FIG. 22. More specifically, data management device 1 may specify, as a processing module to be used for displaying the analysis data, a processing module corresponding to the analysis software specified by this value among one or more processing modules (processing modules 114 to 116 in the example in FIG. 3) stored in storage unit 11. The correspondence relation between one or more pieces of analysis software and one or more processing modules may be stored in storage unit 11, for example, as module information 112C.

Referring back to FIG. 18, in step S32, data management device 1 determines whether or not the operation detected in step S10 is an operation to search the analysis data. If so (YES in step S32), data management device 1 advances the control to step S34, and if not (NO in step S32), it advances the control to step S36.

In step S34, data management device 1 performs a process of searching the analysis data. Then, data management device 1 advances the control to step S36. Data management device 1 receives an operation to search the analysis data, for example, in the manner described with reference to FIG. 16. FIG. 23 is a flowchart of a subroutine of a process of searching the analysis data. The details of step S34 will be described with reference to FIG. 23.

In step S3400, data management device 1 accepts a search condition. In step S3400, data management device 1 may display a list as described as list window 1611 in FIG. 16, and may cause the user to select an item of the search condition from the list.

In step S3402, data management device 1 generates a search key by using the search condition accepted in step S3400.

In step S3404, data management device 1 conducts a search on the analysis data stored in database 15 by using the search key generated in step S3402.

In step S3406, data management device 1 displays the result of the search in step S3404. Then, data management device 1 returns the control to FIG. 18.

Referring back to FIG. 18, in step S36, data management device 1 determines whether or not the operation detected in step S10 is an operation to export the analysis data. If so (YES in step S36), data management device 1 advances the control to step S38, and if not (NO in step S36), it advances the control to step S40. Note that data management device 1 receives the operation to export the analysis data on screen 1400 in FIG. 14 or screen 1700 in FIG. 17.

In step S38, data management device 1 exports the designated analysis data to an external device, and advances the control to step S40.

In step S40, data management device 1 determines whether or not the operation detected in step S10 is an operation other than the above-described operations (to register a new project in S12, to register a new material in S16, to register material information in S20, to register analysis data in S24, to display the analysis data in S28, to search the analysis data in S32, and to export the analysis data in S36). If so (YES in step S40), then in step S44, data management device 1 performs the control corresponding to the details of the operation, and returns the control to step S10. If not, data management device 1 directly returns the control to step S10.

According to the process described above, in the state in which the material is selected, data management device 1 accepts the selection of the analysis data to be registered in database 15 in association with this material. Data management device 1 registers the selected analysis data in database 15 in association with the selected material. When displaying the analysis data, data management device 1 selects a processing module to be used for displaying the analysis data based on the character string included in the file name of this analysis data.

When registering the analysis data in database 15, data management device 1 determines whether or not the analysis data includes an outlier, as described with reference to FIG. 21. When registering the analysis data in database 15, data management device 1 may specify the type of analysis software that processes the analysis data.

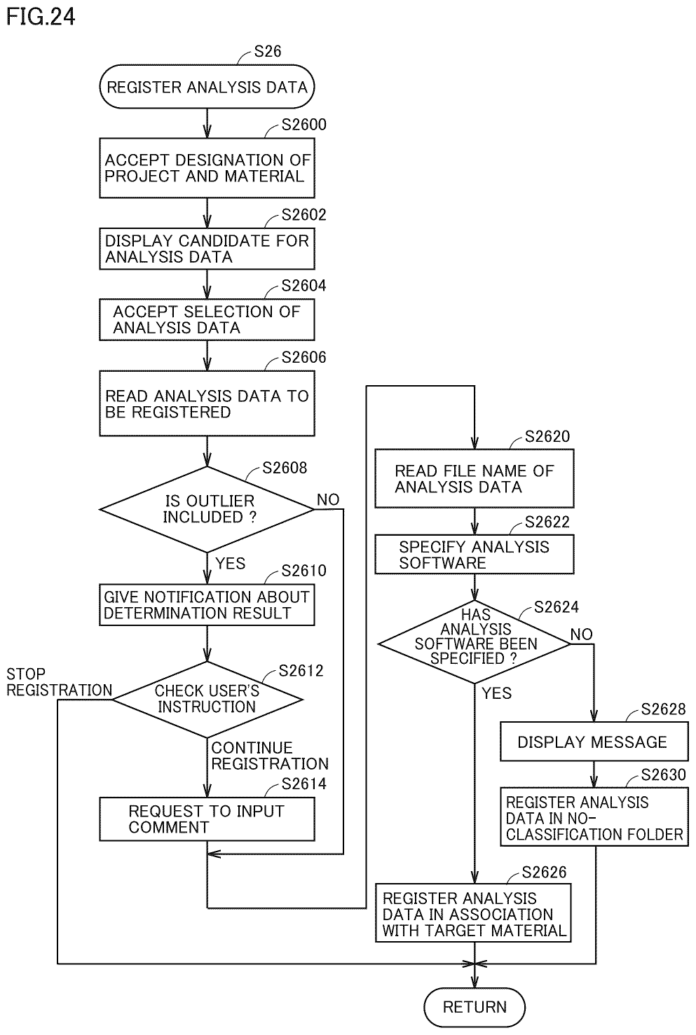

FIG. 24 is a flowchart of a modification of the process in FIG. 21. The flowchart in FIG. 24 includes steps S2620 to S2626 instead of step S2616 in FIG. 21. In the modification shown in FIG. 24, when data management device 1 determines in step S2608 that an outlier is not included in the analysis data (NO in step S2608), it advances the control to step S2620. When data management device 1 determines in step S2608 that an outlier is included in the analysis data (YES in step S2608), it advances the control to step S2610. Then, after data management device 1 requests inputting of a comment in step S2614, it advances the control to step S2620.

In step S2620, data management device 1 reads the file name of the analysis data to be registered (the analysis data selected in step S2604).

In step S2622, based on the file name read in step S2620, data management device 1 specifies the analysis software corresponding to the analysis data to be registered. More specifically, according to the present modification, in module information 112C, the analysis software is associated with the processing module. Data management device 1 searches the file name read in step S2620 for a character string registered in module information 112C. Then, data management device 1 specifies the processing module associated with the character string included in the file name in module information 112C, and specifies the analysis software corresponding to the specified processing module as analysis software corresponding to the analysis data to be registered. The character string to be searched for may be a character string that constitutes an extension of the file or may be a character string that does not constitute an extension of the file.

In step S2624, data management device 1 determines whether or not the analysis software corresponding to the analysis data to be registered has been specified in step S2622. Then, when data management device 1 determines that the analysis software has been specified (YES in step S2624), it advances the control to step S2626, and if not (NO in step S2624), it advances the control to step S2628.

In step S2626, after adding the information about the specified analysis software to the analysis data to be registered, data management device 1 registers the analysis data in database 15 in association with the project and the material specified in step S2600. Then, data management device 1 returns the control to FIG. 18.

In step S2628, data management device 1 displays a message indicating that the analysis software corresponding to the analysis data to be registered cannot be specified and that the analysis data to be registered is registered in a specific folder in database 15. The message is an example of a “warning” given as a notification when the processing module cannot be selected based on the module information and the character string included in the file name of the analysis data. The message may include information (for example, a URL) representing a “specific folder”.

In step S2630, data management device 1 registers the analysis data to be registered in a specific folder (a predetermined folder for unclassified analysis data) in database 15. Then, data management device 1 returns the control to FIG. 18.

In the modification described with reference to FIG. 24, even when the analysis data to be registered does not include the information specifying the analysis software, data management device 1 can specify the analysis software corresponding to the analysis data, and thereby can specify the processing module for processing the analysis data. When data management device 1 cannot specify the analysis software corresponding to the analysis data to be registered, it issues a warning. Thereby, the user can add the information about the analysis software to the analysis data registered in a specific folder. The user can also change the storage location of the analysis data to a folder corresponding to the material corresponding to the analysis data.

In data management system 100, each user can be authorized to display, register, and edit data.

In one example, for a certain project, all users may be permitted to display data, and only the constituent members of the project may be permitted to register and edit data. In another example, only the constituent members of the project may be permitted to display data, and only some of the constituent members of the project may be permitted to register and edit data.

In data management system 100, user authentication may be required for operating data management device 1 or for accessing data management device 1, and each user may be required to input his/her user ID to data management device 1 for user authentication. Thereby, during execution of the management application, data management device 1 can specify the user who is operating data management device 1 or who is accessing data management device 1. Then, in accordance with the details of the authority given to the specified user, data management device 1 can determine whether or not to perform the operation in response to the user's operation to display, register, and edit data. The details of the authority are registered, for example, as project information 112B. The information about the user is registered as user information 112A.

As described with reference to FIGS. 21 and 24, in data management system 100, the user specifies a project and a material when registering the analysis data. Thereby, the analysis data is registered in the folder corresponding to the designated project and material. From the file name of the analysis data to be registered, data management device 1 may specify the source material of the sample having been measured for obtaining the analysis data, and then register the analysis data in the folder corresponding to the specified material. Thereby, data management device 1 can register the analysis data in an appropriate folder in the state in which the project and the material are not designated.

In one example, when data management device 1 receives a request for registration of certain analysis data from the user, it searches the file name of the analysis data for a character string representing any one of the names of one or more materials registered in database 15. When the file name of the analysis data includes a character string representing the name of any one of the materials, data management device 1 registers the analysis data in a folder corresponding to this one material (a folder of the “analysis data” in the folder of this one material). In one implementation, if all the materials have different names in data management system 100 and information processing device 6 is configured to set the file name of the analysis data to include a character string representing the name of the source material of the sample to be analyzed, the user can register the analysis data in an appropriate folder without having to specify a project and a material when registering the analysis data in database 15.

### [Aspects]

It will be understood by those skilled in the art that the above-described exemplary embodiments are illustrative examples of the following aspects.

(Clause 1) A data management device according to one aspect is a data management device that manages analysis data of a material, the data management device comprising: a memory; and a processor accessible to the memory, wherein the memory stores: two or more types of processing modules; and module information including character strings each corresponding to each of the two or more types of processing modules, and the processor may select, among the two or more types of processing modules, for given analysis data, one processing module to be used for processing the given analysis data, based on the module information and a character string included in a file name of the given analysis data.

According to the data management device described in Clause 1, a technique is provided for, in a device that processes analysis data acquired in a plurality of types of analyzers, simply and accurately registering information specifying a program to be used for processing each piece of analysis data.

(Clause 2) In the data management device described in Clause 1, the character string may include a file extension.

According to the data management device described in Clause 2, the program to be used for processing each piece of analysis data is more reliably specified.

(Clause 3) In the data management device described in Clause 1 or 2, the memory stores data of each of two or more materials, and the processor may specify a given material corresponding to the given analysis data from among the two or more materials based on the file name of the given analysis data, and register the given analysis data in the memory in association with the given material.

According to the data management device described in Clause 3, the analysis data can be registered in association with an appropriate material without requiring the user to select a material.

(Clause 4) In the data management device described in any one of Clauses 1 to 3, the processor may issue a warning when the one processing module is unable to be selected based on the character string included in the file name of the given analysis data and the module information.

According to the data management device described in Clause 4, when the processing module to be used for processing the analysis data to be registered cannot be specified from the file name, the user can be caused to reliably take necessary measures.

(Clause 5) In the data management device described in any one of Clauses 1 to 4, the processor may register the given analysis data in a specific area of the memory when the one processing module is unable to be selected based on the character string included in the file name of the given analysis data and the module information.

According to the data management device described in Clause 5, the user can be caused to reliably take necessary measures for the analysis data for which a processing module to be used for processing is not specified.

(Clause 6) A data management method according to one aspect is a method implemented by a computer for managing analysis data of a material, wherein the computer is configured to process the analysis data by using two or more types of processing modules and includes a memory that stores module information including character strings each corresponding to each of the two or more types of processing modules, and the data management method comprises: reading a file name of given analysis data; and, based on the module information and a character string included in the file name, selecting, among the two or more types of processing modules, one processing module to be used for processing the given analysis data.

According to the data management method described in Clause 6, a technique is provided for, in a device that processes analysis data acquired in a plurality of types of analyzers, simply and accurately registering information specifying a program to be used for processing each piece of analysis data.

Although the embodiments of the present invention have been described, it should be understood that the embodiments disclosed herein are illustrative and non-restrictive in every respect. The scope of the present invention is defined by the terms of the claims, and is intended to include any modifications within the scope and meaning equivalent to the terms of the claims.

What is claimed is:

1. A data management device that manages analysis data of a material, the data management device comprising:
a memory; and
a processor accessible to the memory, wherein
the memory stores
two or more types of processing modules; and
module information including character strings each corresponding to each of the two or more types of processing modules, and
the processor is configured to select, among the two or more types of processing modules, for given analysis data, one processing module to be used for processing the given analysis data, based on the module information and a character string included in a file name of the given analysis data.

2. The data management device according to claim 1, wherein the character string includes a file extension.

3. The data management device according to claim 1, wherein
the memory stores data of each of two or more materials, and
the processor is configured to
specify a given material corresponding to the given analysis data from among the two or more materials based on the file name of the given analysis data, and
register the given analysis data in the memory in association with the given material.

4. The data management device according to claim 1, wherein the processor is configured to issue a warning when the one processing module is unable to be selected based on the character string included in the file name of the given analysis data and the module information.

5. The data management device according to claim 1, wherein the processor is configured to register the given analysis data in a specific area of the memory when the one processing module is unable to be selected based on the character string included in the file name of the given analysis data and the module information.

6. A data management method implemented by a computer for managing analysis data of a material, wherein
the computer:
is configured to process the analysis data by using two or more types of processing modules; and
includes a memory that stores module information including character strings each corresponding to each of the two or more types of processing modules, and
the data management method comprises:
reading a file name of given analysis data; and
based on the module information and a character string included in the file name, selecting, among the two or more types of processing modules, one processing module to be used for processing the given analysis data.
